(19) United States
(12) Patent Application Publication
(10) Pub. No.: US 2023/0085686 A1
(43) Pub. Date: Mar. 23, 2023

(54) Multicompartement hydrogel fibre their preparation and uses thereof

(71) Applicant: Association For The Advancement Of Tissue Engineering And Cell Based Technologies & Therapies (A4TEC, Guimarães (PT)

(72) Inventors: Carlos FERREIRA GUIMARÃES, Guimarães (PT); Luca GASPERINI, Braga (PT); Alexandra Margarida PINTO MARQUES, Porto (PT); Rui Luis GONÇALVES DOS REIS, Porto (PT)

(21) Appl. No.: 17/904,530

(22) PCT Filed: Feb. 19, 2021
(86) PCT No.: PCT/IB2021/051425

§ 371 Date: Aug. 18, 2022

(30) Foreign Application Priority Data
Feb. 19, 2020 (PT) 116124
Dec. 29, 2020 (EP) EP 20217710.1

(51) Int. Cl. D01D 5/34; D01F 8/18; D01D 5/06; C12N 11/02; C12M 1/12

(52) U.S. Cl. CPC D01D 5/34; D01F 8/18; D01D 5/06; C12N 11/02; C12M 25/14; D10B 2401/022; D10B 2509/00; D10B 2211/20; C12N 2537/10; C12N 2533/54; C12N 2533/74

(57) ABSTRACT

The present disclosure relates to a hydrogel fibre comprising an ionic hydrogel and a second component in a plurality of compartments, wherein the second component is selected from a second hydrogel, a hydrophilic solution, or a mixture thereof. It is also disclosed the method to obtain the aforesaid hydrogel fibres. This disclosure also relates to a composition comprising the hydrogel fibres and a suitable carrier, and an article/kit, a bundle, a mesh or a membrane comprising the hydrogel fibre. A composition comprising an ionic hydrogel and a second component for use in medicine administered in a hydrogel fibre comprising a plurality of compartments is also disclosed.

### TECHNICAL FIELD

The present disclosure relates to the production of hydrogel fibres, in particular microfibers, with distinct compartments using a flow-focusing system, in particular a single flow-focusing system. The microfibers can integrate distinct types of materials, cells, and molecules. The simple manipulation of processing conditions (as pressure, flow and viscosity of the hydrogels precursors, allows the fabrication of several structures and compartments within the same microfiber, in total diameters down to 50 μm.

### BACKGROUND

These facts are disclosed in order to illustrate the technical problem addressed by the present disclosure.

Hydrogel structures have been widely used in the fields of Tissue Engineering for the encapsulation of cells within a 3D environment.

Fibre-like structures have great importance per-se, but also due to the possibility of assembly into larger size constructs (e.g. in imprinting). Fibres can be formed by separate materials recognizable in the cross-sectional area, and the inclusion of different materials within such structures has been done by co-axial extrusion/needle systems, where a material is included within another or by microfluidic systems where channels are arranged to obtain similar flow conditions.

Frequently, up to 3 or 4 maximum layers of distinct materials can be organized in a co-centric fashion forming co-centric shapes in the cross-sectional area. This kind of fibre usually requires a system of needles/channels organized in a co-axial manner (outer surrounding inner) in which the channel size or needle diameter limits the size of the produced fibre as well as the versatility of the systems.

Often, obtaining fibres with distinct sizes implies a full change in the used fabrication setup. Core-shell hydrogel microfibers reported in the literature have a minimum size of 100 μm (core+shell), being frequently larger than 500 μm.

One other shape that exists is the Janus shape, where 2 materials flow side-by-side forming a fibre. The cross-section of a Janus fibre is half composed of one material and half of the other material, forming a shape of a circle divided in two. Janus structures have also been combined as a fibre core surrounded by one other shell or crosslinker material.

While core-shell and Janus systems that exist function quite well, they are limited by the fact that manipulation of sizes frequently requires a change in the fabrication setup, making it complex and not versatile.

The shapes that can be obtained by the state-of-the-art systems are at most combinations of coaxial and Janus, using a specialized microfluidic chip or needle setup for each of the shapes, in order to obtain co-centrical or parallel structures. The use of Janus and coaxial shape is limited to applications where those shapes are meaningful.

For some other applications, fibres with different shapes are needed. Such fibres have important value due to the possibility of obtaining selected shapes that mimic those of important biological structures such as blood vessels and stroma/cancer cell interaction. It is not possible to obtain other shapes than coaxial and Janus from standard commercial chips, and a new microfluidic chip has to be designed and manufactured for each scenario, with increased associated costs. Furthermore, some shapes may not be possible even with specialized chips because they are constituted by very thin regions of material and/or particularly complex shapes.

Existing in vitro screening technologies such as organ-on-chips mainly employ chip-circumscribed structures, where several chips have to be used in order to evaluate different conditions, which can become considerably expensive and time consuming. Also, in many organ-on-a-chip systems, 2D environments are still used for cellular behaviour screening, which is already known to yield limited responses.

Organ-on-a-chip solutions can recapitulate the human physiology, in some cases going up to 10 simultaneous organs. Nevertheless, these chip-circumscribed structures are not only expensive to fabricate but also sometimes use 2D environments to represent tissues/organs.

Core shell fibres are currently widely used within different applications, but this configuration was not established as a product for in vitro screening yet.

Core-shell fibres are employed to fabricate vascular-like structures, but their methods of fabrication limit their introduction within 3D constructs and as such are not yet established as a tissue engineering therapy.

General multi-compartment fibres are widely used to fabricate uniform tissue structures, but they are not able to be used as a platform to simultaneously carry stem cells and pro-differentiation hydrophobic compounds as an all-in-one tissue engineering approach.

The document WO2011046105 A1, relates to gel microfibres with improved mechanical strength. The microfibre is composed by a microgel fibre material coated with a high-strength alginate hydrogel, resulting in fibre with a core-shell structure. The fibres are obtained using a co-axial microfluidic device, and the final diameter of the fibres can be modulated within a range of 200 nm to 2000 μm. The resulting fibres have two distinct compartments, but the cross-sectional shape does not change along the fibre length axis. Also, different shapes are only achieved at the macroscopic level, and using braiding techniques.

The document WO2015178427 A1, discloses a hollow concentric core-shell microfibre, including a cell-adhesive hydrogel covered by a high strength hydrogel layer. The fibre is obtained using an apparatus comprising three co-axial tubes with distinct inlets and a shared outlet. The resulting fibre has a diameter ranging from 20 to 500 μm. Nevertheless, configurations different from the hollow concentric core-shell morphology are not disclosed.

CN106215987 B from, relates to a multi-channel co-current microfluidic chip composed of at least three shunt capillaries. The microfluidic chip can be used in conventional wet and dry spinning processes, but also for electrospinning. Additionally, the microfluidic chip allows the production of linear heterogeneous multi-structure fibres with a diameter ranging from 30 to 1000 μm. The multiple-structure fibres can be obtained by changing the number of the capillary tubes or by dosing the inlets of the multi-channel microfluidic chip. Regardless, the disclosed invention only refers to fibres prepared using a co-axial flow.

The document US20160068385, relates to the methods of use of a microfluidic device, aiming the controlled formation of tubular structures, whose diameter is greater than 1000 μm. The method allows a controlled and continuous extrusion of tubular structures with tailored heterogeneities, as well as predictable mechanical and chemical properties. Nevertheless, the invention only relates to tubular structures, thenceforth not allowing the preparation of compact hydrogel fibres.

WO2018162357 A1, relates to a method to prepare a hollow microfibre, comprising concentric cell layers, an extracellular matrix layer and an optional hydrogel outer layer. The method allows the production of fibres with different dimensions, with an external diameter between 70 μm and 5 mm. Again, only the tubular shape is compatible with the disclaimed production method.

### GENERAL DESCRIPTION

The present application relates to a multi-compartment hydrogel fibre comprising at least two components, wherein at least one of the components is an ionic hydrogel. The disclosure also provides a method to prepare the multi-compartment fibres, which are obtained using a single setup, and the structure of the fibres can be changed during production, in real-time.

In the present disclosure, a hydrogel is a network of polymer chains that are hydrophilic, sometimes found as a colloidal gel in which water is the dispersion medium. A three-dimensional solid results from the hydrophilic polymer chains being held together by cross-links. Because of the inherent cross-links, the structural integrity of the hydrogel network does not dissolve from the high concentration of water. Hydrogels are highly absorbent (they can contain over 90% water) natural or synthetic polymeric networks.

In the present disclosure, an ionic hydrogel/an ionic crosslinkable hydrogel is a hydrogel which forms upon the combination of a hydrogel precursor (polymeric solution) with ions, which will interact with and bind the polymeric chains.

In an embodiment, multi-compartment fibres are prepared using a 3D flow-focusing microfluidic chip, combined with the use of a pressure regulator. When applying a flow focusing condition to hydrogel precursors and posterior crosslinking of the material, it is possible to obtain fibres with multi-shapes/compartments and different organization by simply controlling viscosities and flows, and allowing the material to arrange by the consequences of flow focusing through a 3D pore.

In an aspect of the present disclosure, the shape of these fibres can be maintained even with significant diameter size reductions. The method disclosed on the present subject-matter allows the fabrication of full core-shell structures below 50 μm in diameter. This brings the size of the produced structures close to the size magnitude of single cells and, e.g., capillary blood vessels. Nevertheless, large-diameter structures can still be produced using the same setup.

In an embodiment, the method of the present disclosure permits the use of a single setup to obtain distinct structures; the ability to change the size of the structure's compartments during production in real-time; the integration of different materials and crosslinking mechanics within a same structure.

In yet another embodiment, the present disclosure allows the formation of multi-shape hydrogel fibres, not only the known core-shell and Janus structures but also novel shapes which have never been reported, with the possibility to manipulate sizes and geometries in real time by changing flow conditions. This can yield important structures such as core shell fibres, but also novel shapes with biomimicry relevance henceforth named as ribbon, dual core-shell, double Janus, tricoaxial, oil-core-hydro-shell, among others.

Furthermore, the present subject-matter allows the integration of distinct cell types and materials in different yet connected compartments for in vitro disease modelling (e.g. cancer-stroma interactions), as well as the possibility to transport not only cells but also depots with specific insoluble molecules to direct their responses, such as stem-cell differentiation in an all-in-one approach.

The present disclosure also relates to a method to obtain multi-compartment hydrogel fibres, wherein the structure of the fibres is changed during production, in real-time, and using a single setup.

In an embodiment, the present disclosure relates to a hydrogel fibre, in particular multi-compartment hydrogel fibre wherein the fibre has an outer and an inner layer, comprising an ionic hydrogel and a second component in a plurality of compartments, wherein the second component is selected from a hydrophobic solution, a second hydrogel, a hydrophilic solution, or a mixture thereof. The outer layer of the fibre comprises an ionic hydrogel and the ionic hydrogel and the second hydrogel have different compositions, provided that if the second component is a second hydrogel the compartments are axially nonconcentric/off-centred.

The present invention relates to a multi-compartment hydrogel fibre wherein the fibre has an outer and an inner layer comprising:
a first ionic hydrogel and a second component in a plurality of compartments,
wherein the second component is selected from a second hydrogel, a hydrophilic solution, or a mixture thereof;
wherein the outer layer of the fibre comprises the ionic hydrogel; and
the ionic hydrogel and the second hydrogel have different compositions, provided that if the second component is a second hydrogel:
the compartments are axially nonconcentric/off-centred; or the cross-section of one of the plurality of compartments is not circular, or the fibre comprises an equivalent diameter inferior to 200 μm.

In an embodiment, the multi-compartment hydrogel fibre of the present disclosure comprises an ionic hydrogel and a second hydrogel wherein the compartments are axially nonconcentric/off-centred.

In the present disclosure nonconcentric signifies that the compartments do not have a common centre or the compartment is situated away from the centre or axis of the fibre (off-centre).

In an aspect of the present embodiment, each compartment is delimited by the boundaries between at least two different components of the fibre, or the boundaries between at least two different components and the external environment of the fibre.

In an embodiment, the ionic hydrogel is selected from a list consisting of: gellan gum, alginate, chitosan or mixtures thereof; preferably gellan gum, alginate or mixtures thereof. The second hydrogel is selected from a list consisting of: gellan gum, alginate, acid hyaluronic, gelatin, basement membrane extract, collagen, fibrin, biological lysates, silk solutions, dextran solutions, polyethyene glycol, chitosan, heparin, acrylamide, starch, cellulose, guar gum, xanthan gum or mixtures thereof; preferably gellan gum, alginate, acid hyaluronic, gelatin, basement membrane extract, or mixtures thereof.

In a further embodiment, the second hydrogel is a photo-crosslinkable hydrogel.

In an embodiment, the hydrogel fibre further comprises an additional compartment, in particular an additional compartment comprising a third hydrogel, a fourth hydrogel or further hydrogel.

In an embodiment, the ionic hydrogel is a gellan gum hydrogel, preferably dissolved in 0.15M to 0.30M aqueous sucrose solution.

An aspect of the present disclosure relates to fibres with an equivalent diameter inferior to 200 μm, preferably between 50 μm and 170 μm.

In an embodiment, the hydrogel fibre of the present subject-matter may comprise an ionic hydrogel and a second hydrogel wherein the compartments are axially nonconcentric/off-centred. The cross-sectional area of the fibres related to the present embodiment comprise the following shapes: core-shell; or ribbon; or tricoaxial; or double-Janus; or double core-shell.

The cross-sectional area is the area of a two-dimensional shape that is obtained when a three-dimensional object—such as a cylinder—is sliced perpendicular to some specified axis at a point. For example, the cross-section of a cylinder fibre—when sliced parallel to its base—is a circle.

In another embodiment, the hydrogel fibre may comprise an ionic hydrogel and a hydrophobic solution, wherein the outer layer of the fibre is the ionic hydrogel. The hydrophobic solution and the hydrogel compartments are axially concentric. In an aspect of the present embodiment, the hydrophobic solution is confined to spherical compartments inside an ionic hydrogel shell. In an embodiment, the hydrophobic solution is a suitable oil, preferably an oil with pharmaceutical grade, more preferably an oil selected from sesame oil, mineral oil, soybean oil, castor oil, essential oil, or mixtures thereof.

In an embodiment, the hydrogel fibre further comprises an anti-inflammatory agent, an antiseptic agent, an antipyretic agent, an anaesthetic agent, a therapeutic agent, a cell, or combinations thereof. The cell may be a non-human animal cell, or human cell, or stem cell, or combinations thereof.

In an embodiment, the method to prepare the hydrogel fibres comprises: (i) injecting the ionic hydrogel precursor and the second component solution into the channels of the flow focusing microfluidic chip, wherein the second component solution and the ionic hydrogel precursor have a distinct viscosity at 25° C.; (ii) applying variable pressure to the channels of the microfluidic chip by the action of a pressure regulator in order to obtain a hydrogel fibre precursor; and (iii) obtaining the hydrogel fibre by extruding the hydrogel fibre precursor into an ionic cross-linking bath/solution, wherein the ionic solutions are selected from solutions with positive ions, such as Na⁺, K⁺, Ca²⁺, Mg²⁺, Ba²⁺ or Sr²⁺, selected from calcium chloride, cell culture medium, calcium sulphate, calcium carbonate, phosphate buffer saline, preferably calcium chloride solutions with a concentration between 0.01-5M, preferably 0.01-0.2M.

In a further embodiment, the hydrogel fibre is further crosslinked by light, preferably light with a wavelength ranging between 320 to 500 nm, during 30 to 60 seconds, and using an energy flux between 0.5 to 0.7 mW/cm².

In an embodiment, the hydrogel precursors may have a shear viscosity at 25° C. between 0.01 to 100 Pa·s, preferably between 0.1 to 10 Pa·s.

In an embodiment, when the second component of the fibre is a hydrogel, the shear viscosity of the second hydrogel precursor is from 2-1000 times higher than the shear viscosity of the ionic hydrogel component, preferably the shear viscosity of the second hydrogel precursor is from 10-100 times higher than the shear viscosity of the ionic hydrogel component; even more preferably the shear viscosity of the second hydrogel precursor is from 10-50 times higher than the shear viscosity of the ionic hydrogel component.

In an embodiment, the hydrogel precursor is dissolved at a concentration between 0.25 wt % to 10 wt %, preferably 0.5 wt % to 1 wt. %.

In an embodiment, the flow focusing microfluidic chip comprises a plurality of channels, namely 2, 3, 4, 5 channels. In the present disclosure, the channels of the microfluidic chip are divided as outer and inner channels, wherein the outer channels relate to the most external channels of the chip and the inner ones to the channels located in between the outer channels (as viewed from a top view).

In an embodiment, the pressure applied in one channels is independent to the pressure applied in another channel.

In an embodiment, the applied inner pressure varies from 10 to 800 kPa, preferably between 15-60 kPa. On the other hand, the applied outer pressure varies from 15 to 800 kPa, preferably from 15-60 kPa.

In an embodiment, the microfluidic chip comprises 4 channels, and the pressure applied to the outer channels is equal or greater than the pressure applied into the inner channels.

In a further embodiment, the method to prepare an oil-core-hydro-shell hydrogel fibre requires that the inner channels of the microfluidic chip are filled with a hydrophobic solution.

A yet another embodiment relates to a composition comprising the hydrogel fibres combined with a suitable carrier, wherein the carrier is any 3D material, cell suspension, tissue engineering construct, or combinations thereof.

In an embodiment, this disclosure relates to a composition comprising an ionic hydrogel and a second component for use in medicine administrated in a hydrogel fibre comprising a plurality of compartments, wherein the second component is selected from a hydrophobic solution, a second hydrogel, a hydrophilic solution, or a mixture thereof; wherein the outer layer of the fibre comprises the ionic hydrogel; and the ionic hydrogel and the second hydrogel have different compositions, provided that if the second component is a second hydrogel the compartments are axially nonconcentric/off-centred.

In an embodiment, the present disclosure relates to an article/kit comprising the hydrogel fibres disclosed in the previous embodiments, wherein the article/kit is a multi-compartment medical-device, preferably a cell carrier, therapeutic hydrogel, drug delivery depot, or combinations thereof.

In an embodiment, the present disclosure also comprehends a bundle, a mesh or a membrane comprising the hydrogel fibres described in any of the previous embodiments.

In an embodiment, the use of the hydrogel fibre as in vitro vasculature model, in vitro tumour model, in vitro multi compartment tissue model, high throughput testing platform, or mixtures thereof is also disclosed.

The technology related to the present disclosure allows a faster and cheaper method to prepare fibres with multi-shapes/compartments, using different materials and cells. Produced structures are fully 3D structures which are not limited to any area but rather free to be manipulated or subjected to further conditions. The full structure may use materials different than those naturally present in tissues but lately the whole outcome will be dependent only on cells and their environment, in 3D.

### BRIEF DESCRIPTION OF THE DRAWINGS

The following figures provide preferred embodiments for illustrating the disclosure and should not be seen as limiting the scope of invention.

FIG. 1: Schematic representation of an embodiment of the dimensions of a flow focusing chip.

FIG. 2: Schematic representation of an embodiment using different flow configurations for fabricating distinct shapes. A) Core-Shell Shape, B) Ribbon Shape, C) Oil Core in Hydrogel Shell Shape, D) Tricoaxial Shape, E) Double Janus, F) Double Core-Shell, Hydrogels are represented according to the indicated colour code: Black for the More Viscous gellan gum (GG), Wavy for the less viscous GG, wavy-checker for a third GG of intermediate viscosity present in tricoaxial shape. Grey represents channels which are blocked (not having any flow). Sand patterns indicate a hydrophobic solution (oil).

FIG. 3: Embodiment of relative compartment size control: changing the inner/outer pressures leads to differences in compartment size, here shown with core-shell (A) and ribbon (B) shapes. bGG—GG with blue microparticles (less viscous), rGG—GG with red microparticles (more viscous). Scale bars: 100 μm.

FIG. 4: Embodiment of other fibre shapes obtained by manipulating the hydrogel precursors at extreme flows which will lead to bending of compartments as well as leaking inner-to-outer flows (A).

FIG. 5: Embodiment of the impact of different hydrogel viscosity on the final fibre shape. When the more viscous red-GG flows in the inner channels, the ribbon can be obtained as the less viscous blue-GG surrounds the inner material and compresses it into the ribbon shape (A). Reversing the materials and therefore also the viscosities leads to the loss of ribbon shape, as the inner, less viscous material tends to squeeze around the outer, more viscous counterpart (B). Grey—GG with blue particles, Dark Grey—GG with red particles, Light Grey—Blocked channel.

FIG. 6: Representation of an embodiment showing the importance of applied pressure on final compartment dimensions, as the dimension of compartments along the same fibre can be controlled be changing the applied pressure along time (A). Outer/Constant: blue GG, Inner/Changing: red GG. Experimentally measured diameters (from fibre centre) were compared to programmed (estimated) ones showing that it is possible to translate input functions to the final structure (B).

FIG. 7: Embodiment of fibre size reduction. Fibre spinning upon crosslinking can gradually reduce the size of the fibres without compromising shape, as demonstrated with the core-shell structure, reaching full diameters below 50 μm. bGG—GG with blue microparticles (less viscous), rGG—GG with red microparticles (more viscous). Scale bars: 100 μm unless stated.

FIG. 8: Embodiment of microscopy images of flow focusing fibres axial cuts: A) Core-Shell Shape, B) Ribbon Shape, C) Oil Core in Hydrogel Shell Shape, D) Tricoaxial Shape, E) Double Janus, F) Double Core-Shell, bGG—GG with blue microparticles (less viscous), rGG—GG with red microparticles (more viscous), GG (pure GG, intermediate viscosity). Scale bars: 100 μm.

FIG. 9: Embodiment of size and distribution manipulation of oil-core hydrogel-shell fibres: A—Fibres obtained with oil flowing at a similar pressure to that of GG. B—Fibres obtained while gradually reducing the pressure of oil flow.

FIG. 10: Embodiment of production of flow focusing fibres using alginate. Similar shapes can be produced using Alginate, as visible on the axial (A) and longitudinal (B) cuts of ribbon-shaped alginate fibres. bAlg—Alginate with blue microparticles, rAlg—Alginate with red microparticles. Scale bars: 100 μm.

FIG. 11: Embodiment of fibres prepared using non-ionic materials blended with ionic materials. A GG/gelatin-methacrylate (GelMA) blend is used as shell material, still allowing to form core-shell fibres with tuneable sizes (e.g. shell thickness) (A). The GelMA component allows to have a second crosslinking step with UV light which can be employed to fuse the shells of adjacent fibres in a bottom-up approach (B). Scale bars: 100 μm.

FIG. 12: Embodiment of core-shell fibres formed with ionic and non-ionic hydrogels, wherein non-ionic hydrogels are GelMA and hyaluronic acid (HA). Fibres are formed using a non-ionic, biolabile hydrogel core (GelMA/HA) within the GG shell (A). Scale bar: 200 μm. The mechanical characterization of both core and shell hydrogel materials at the bulk level (B). Viscosity profiles of Gellan Gum (GG), Hyaluronic Acid (HA), and the GelMA/HA blend (C). GelMA 5 wt % is represented as a continuous dashed line as its water-like viscosity is too low to be properly characterized by the rheometer. As can be observed, dissolving GelMA in HA allows increasing its viscosity and overcome that of GG, therefore facilitating the formation of core-shell structures.

FIG. 13: Embodiment of a ribbon shaped fibre. Ribbon shaped fibbers with GG on the outer ribbons and Geltrex (basement membrane (BM) derivative) visible in the axial cut (A) or longitudinally (B). Scale bar 200 μm.

FIG. 14: Representation of an embodiment related to tri-material ribbon fibres. Tri-Material Ribbons can simultaneously include different materials manipulated at distinct temperatures (A). These still allow to obtain the 2 outer compartments separated by a third one and allow for size manipulation such as creating a thicker (B) or thinner (C) Geltrex BM (dashed lines), Scale bars: 200 μm.

FIG. 15: Schematic representation of an embodiment related to the application of the flow focusing fibres. Core-shell flow focusing fibres are suitable for the fabrication of vascular-like structures.

FIG. 16: Embodiment of core-shell fabrication of vascular like structures: Endothelial cells (identified with the endothelial marker CD31) can be encapsulated within the core material (A). Construct maturation for up to 14 days leads to the appearance of primitive lumen-like structures (B). Dashed and dotted lines represent the shell and core limits, respectively. Scale bars: 100 μm.

FIG. 17: Representation of an embodiment related to the use of flow focusing fibres as mimics of vascular structure. Free-form core-shell fibres can be included within a 3D environment containing other materials and cells, in order to obtain a vascular structure within a larger construct (A). B—Randomly oriented core-shell flow focusing fibre placed within a third hydrogel. C—Core-shell fibre containing endothelial cells within a collagen hydrogel populated by fibroblasts (brightfield left, immunocytochemistry on the right). Scale bars; 5 mm (B), 200 μm (C).

FIG. 18: Schematic representation of an embodiment using ribbon flow focusing fibres as 3D cancer models. Distinct materials can be integrated in the structure, e.g. the degradable and adhesive GG/GelMA for the cancer compartment to allow cancer cells to move and a more stable (GG) material for the stromal compartments, to keep fibroblasts in place and study mostly the cancer cell responses. Between both, a thin BM-like compartment can be placed.

FIG. 19: Embodiment of a cancer/BM/stroma model, comprising a ribbon fibre structure with the inclusion of relevant cells such as melanoma (cancer) and fibroblasts (stroma) separated by a BM structure (GelTrex, dotted lines) (A). After 1 day of culture it is possible to observe melanoma cells protruding into the BM (i) and clearly invading through it upon 5 days of culture (ii). Flow focusing configuration allows for a modular deconstruction of the model, by changing the materials/cells flowing, leading to a cancer/stroma structure (B, no BM), or single-stroma (C) and single cancer (D) fibres. Scale bars: 100 μm except Ai and Ali: 50 μm.

FIG. 20: Embodiment using the ribbon flow focusing fibres as a cancer/BM/stroma model for drug testing. Using markers for specific cell responses such as viability, together with cell trackers, it is possible to visualize the presence of death and live cells of each type after a treatment with doxorubicin (A). Scale bars: 50 μm. This can be quantified in order to understand how drugs affect the viability of cancer cells in 3D environments of distinct complexity (B). Cancer Alone—Cancer cell viability in cancer-only fibres. Stroma Alone—Stromal Cell Viability in stroma-only fibres. Cancer (+stroma)—Cancer Cell viability in cancer/stroma fibres. Stroma (+Cancer)—Stromal Cell viability in cancer/stroma fibres. Cancer (+BM Stroma)—Cancer Cell viability in Cancer/BM/Stroma fibres. Stroma (+BM Cancer)—Stroma Cell Viability in Cancer/BM/Stroma fibres.

FIG. 21: Standard 2D culture of melanoma and melanoma/fibroblast co-culture to be used as comparative data. Calcein AM stained cells show live cells with or without doxorubicin treatment. 2D culture systems fail to inform on the complex behaviour of cancer cells. The lack of shape and orientation fails to inform on anything other than eventual live cell numbers upon drug treatment. Scale bars 200 μm.

FIG. 22: Schematic representation of an embodiment using oil droplet core within a hydrogel fibre containing stem cells. As represented, the fabrication of such a structure can simultaneously transport the encapsulated stem cells with hydrophobic molecules that can be used to direct differentiation.

FIG. 23: Embodiment of oil core hydrogel shell fibres. Oil droplets were used as reservoir of fluorescent dexamethasone (dexamethasone—FITC) inside a GG hydrogel fibre (A). If left in a phosphate buffer saline (PBS), the fluorescent molecule is released from the fibre (B). Scale bars: 100 μm. The release profile shows a gradual release of dexamethasone-FITC for a period of around 12 h (C). It is possible to see that the dispersed dexamethasone present in the oil immediately post-fabrication (0h) is no longer visible after 1 day of incubation (24 h). Scale bars: 50 μm.

FIG. 24: Embodiment of cellular responses after encapsulation within oil core hydrogel fibres. After 3 days of culture, stem cells alone in normal medium (control), medium with soluble dexamethasone (medium) or together with oil droplets containing dexamethasone dispersion (oil) were stained against Runx2 together with actin and nuclei (DAPI) (A) Scale bars: 100 μm. Image quantification of thousands of single-cell Runx2 events show a significant increase in Runx2 expression by stem cells cultured with oil droplets releasing dexamethasone when compared to the presence of soluble dexamethasone m the medium (B).

### DETAILED DESCRIPTION

The present disclosure relates to a hydrogel fibre, comprising an ionic hydrogel and a second component in a plurality of compartments, wherein the second component is selected from a hydrophobic solution, a second hydrogel, a hydrophilic solution, or a mixture thereof. Moreover, a method to obtain the hydrogel fibres of the present subject-matter is also encompassed. At last, a composition comprising the hydrogel fibres for use in medicine as well as the use of such fibres as multi-compartment in vitro model are also disclosed.

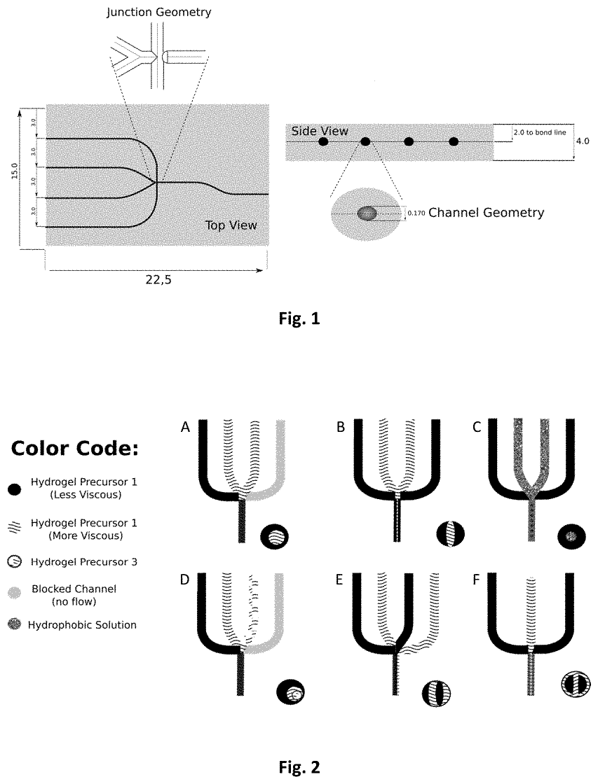

For the fabrication of multi compartment hydrogel fibres, mostly one single chip geometry was used (3D Flow Focusing, Dolomite), whose geometry is represented in FIG. 1. In the present disclosure, the channels of the microfluidic chip are divided as outer and inner channels, wherein the outer channels relate to the most external channels of the chip and the inner ones to the channels located in between the outer channels (as viewed from a top view). Distinct shapes were then obtained by the manipulation of channel blockage, flows, and material viscosity, as schematically represented in FIG. 2A-E.

In an aspect of the present disclosure, specific ranges of pressure had to be applied to the distinct fluids for obtaining the described shapes (Table 1). The values shown in Table 1 illustrate the preferred conditions to obtain the fibres described in the previous embodiments. By varying them closely to the original values, the shape can be maintained, and the relative sizes of the distinct compartments can be manipulated. Certain shapes require differences either in pressure (flow) or viscosity (relatively, between the materials), identified in Table 1 as “limits”.

Table 1 summarizes the conditions required for the fabrication of distinct fibre shapes and sizes.

| | | Inner | Outer | | |
|---|---|---|---|---|---|
| | Chip | Pressure | Pressure | | Viscosity limits |
| Shape | Configuration | (kPa) | (kPa) | Pressure (Flow) Limits | (relative) |
| Core-Shell | 3D FF, 1 outer | 10-15 | 15 | Outer >= Inner. 18 outer | Inner > Outer |
| (A) | channel blocked | | | 15 inner - upper limit at | |
| | | | | which core-shell still | |
| | | | | forms. | |
| Ribbon (B) | 3D FF | 30-60 | 60 | Outer >= Inner. Varying | None. More |
| | | | | Changes relative | Stable flows if |
| | | | | diameters | Inner > Outer. |
| Oil-Core | 3D FF | 10-30 | 30 | Outer >= Inner. Varying | None. |
| (C) | | | | changes the size and | |
| | | | | concentration of oil | |
| | | | | droplets. | |
| Tricoaxial | 3D FF, 1 outer | 10-15 | 15 | Same as Core-Shell but | Inner > Outer |
| (D) | channel blocked | | | distinct materials on the | Core > 2^{nd} Layer |
| | | | | inner 2 channels | |
| Double | 3D FF | 30-60 | 60 | Same as Ribbon but | None. |
| Janus (E) | | | | alternating the materials | |
| | | | | on the channels | |
| Double | FF (2D) | 40-80 | 40-80 | Overall the shape is | None. |
| Core-Shell | | (red inside) | (red inside) | maintained across | |
| (D) | | 40-50 | 40-50 | different flows. | |
| | | (blue inside) | (bine inside) | Changing ratios will | |
| | | | | affect the thicknesses. | |

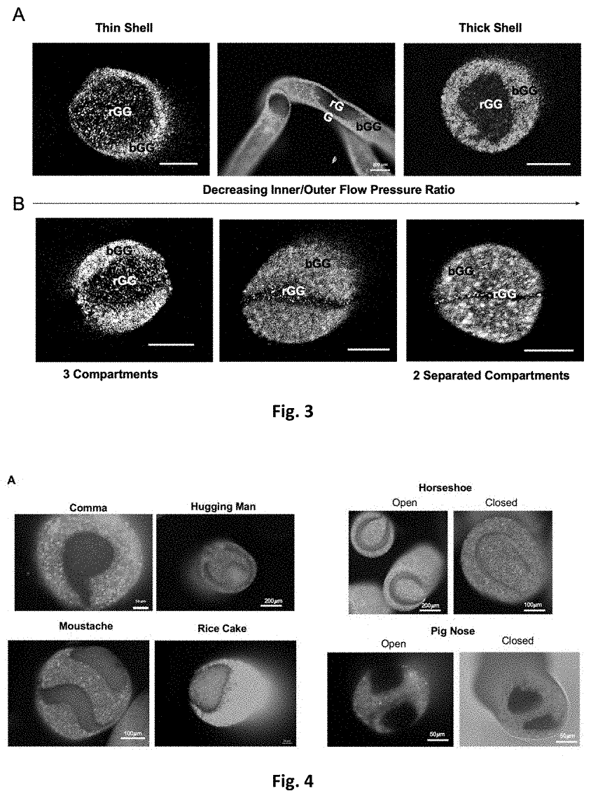

An aspect of the present disclosure comprises the control of relative size compartment. As illustrated in FIG. 3, the relative size of distinct compartments can be manipulated while maintaining the cross-sectional shape. In an embodiment, the thickness of core-shell fibres varies with changes in the inner/outer flow pressure ratios. Fibres produced using an inner/outer flow pressure ratio between 1-1.5 have a thin shell, which can be increased by decreasing the inner/outer flow pressure ratio (FIG. 3A). The same goes to ribbon-shaped fibres, where the relative size of the external compartment increases as the inner/outer flow pressure ratio decreases from 1 to 0.5 (FIG. 3B).

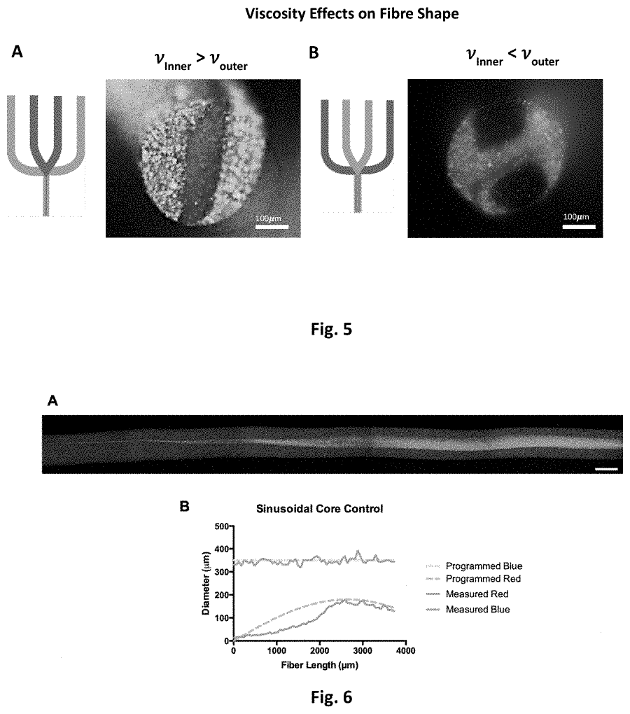

Further shapes can be obtained by manipulating the hydrogel precursors at extreme flows, which will lead to bending of compartments as well as leaking inner-to-outer flows (FIG. 4). Also, when the more viscous hydrogel precursor flows in the inner channels, the ribbon can be obtained as the less viscous blue-GG surrounds the inner material and compresses it into the ribbon shape (FIG. 5A). Reversing the materials, and therefore also the viscosities, leads to the loss of ribbon shape, as the inner, less viscous material tends to squeeze around the outer, more viscous counterpart (FIG. 5B). In the state of the art, the viscosity may be measured by many methods. In the present disclosure, shear viscosity was measured using a Malvern Kinexus Pro+ Rheometer, coupled with a cone plate geometry (40 mm/4° ). Shear viscosity was recorded along a range of 0.1-1000 s⁻¹ of shear rate, at 25° C.)

An aspect of the present disclosures relates to the possibility to program dimension changes along the fibre, while it is being produced. By programming the pressure applied to follow a time-changing function (e.g. sinusoidal), it is possible to alter the dimension of compartments along the same fibre (FIG. 6A). In this example, a constant shell material pressure was maintained, and the pressure applied to the core flow in a sinusoidal fashion led to gradually increasing core diameters along the structure (FIG. 6B). Experimentally measured diameters (from fibre centre) were compared to programmed (estimated) ones showing that it is possible to translate input functions to the final structure.

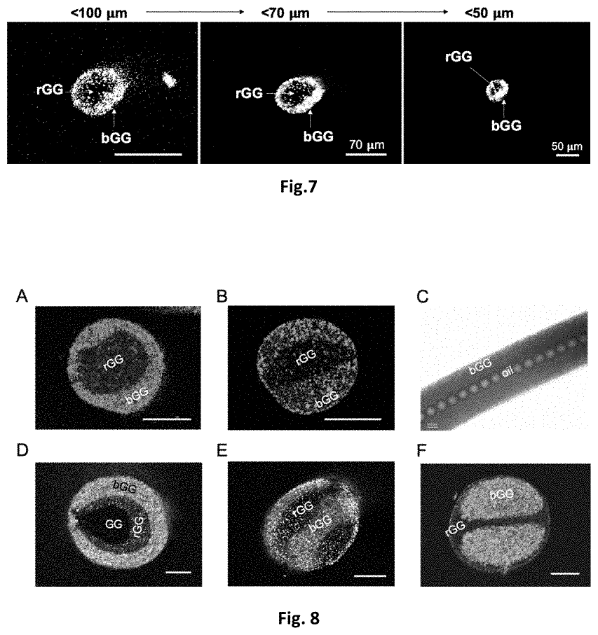

The present disclosure also relates to a further reduction of fibre equivalent diameter. In an embodiment, fibres with the same geometry but smaller equivalent diameters can be obtained by using a 3D flow focusing chip with the same geometry (FIG. 7) but with 100 μm inner channel size (instead of 170 μm). This smaller chip led to the immediate fabrication of smaller diameter fibres, which were then further spun (manually) in order to produce fibres with less than 50 μm of diameter, where the full structure (core-shell) could still be maintained (FIG. 7).

The description of this disclosure is complemented through the following examples that are intended to provide a better understanding of the same, although these examples should not be addressed with a restrictive nature.

### EXAMPLE 1

### Gellan Gum-Based Fibres

In an embodiment, gellan gum (Gelzan, Sigma) was used as the main hydrogel material. Gellan gum (GG) was dissolved at 0.5 wt % in water containing 0.25M sucrose. For contrasting and colouring, GG was mixed with red or blue magnetic microparticles (screenMag, Chemicell), 1:10 particle dilution, or used alone without colour. The interaction between screenMags and GG led to a significant difference in viscosity: red mags increased GG viscosity whereas blue mags reduced it. This difference in viscosity was exploited with flow-focusing conditions to build structures with inner, more viscous components and outer, less viscous ones.

Core-shell fibres could be produced by blocking the flow of one of the outer channels, allowing the inner flow to be surrounded by the outer one (FIG. 1A). For that, the outer hydrogel should be less viscous than the hydrogel precursor that is flowing in the inside channels. As result, the less viscous hydrogel precursor is able to surround the more viscous hydrogel, thus forming the core-shell fibres.

Considering the different viscosities of GG hydrogel precursors labelled with blue or red magnetic microparticles, these solutions were used to prepare the core-shell fibres related to this embodiment. As showed in FIG. 8A, the cross-section of the produced fibres has two compartments arranged in a core-shell fashion, where the blue GG (less viscous) surrounds a red-labelled GG hydrogel (more viscous).

By flowing the more viscous red-labelled GG in the two inside channels, and the less viscous blue-labelled GG in the two outer ones (FIG. 1B), it was possible to form hydrogel fibres with three compartments, in a ribbon-like shape, as showed in FIG. 8B.

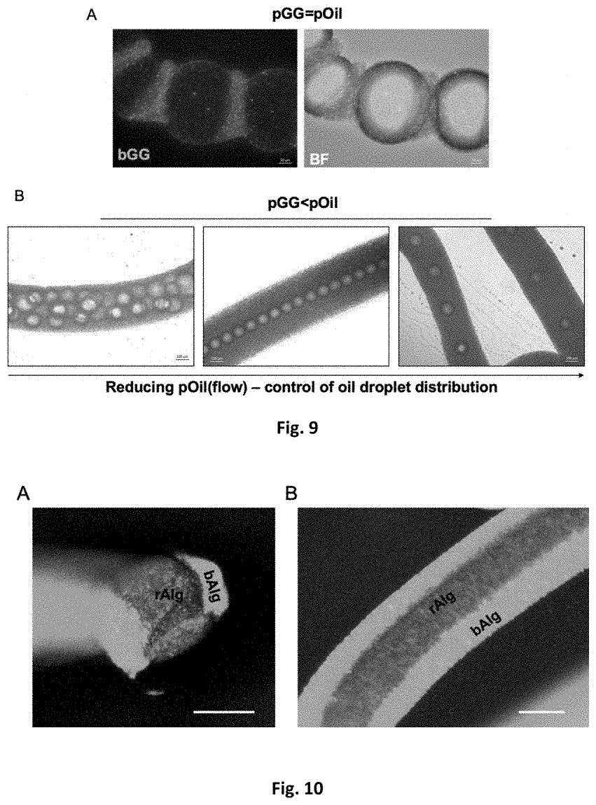

The flow of a hydrophobic solution into the two inside channels combined with the flow of blue-labelled GG in the two outer ones (FIG. 1C) produces a fibre comprising a core of oil droplets within the hydrogel shell (FIG. 8C). Size and distribution of oil droplets within the oil-core hydrogel-shell fibres can be manipulated by changing the flowing pressure ratio between oil and hydrogel. By flowing oil at a similar pressure to that of GG, large oil droplets can be formed encapsulated by a thin layer of hydrogel (FIG. 9A). Gradually reducing the pressure of oil flow leads to a decrease in oil droplet diameter and distribution along the fibre (FIG. 9B).

Tricoaxial-like fibres, comprising three layers of near-co-centrical materials can be fabricated by flowing two materials with different viscosity (GG and red-GG) in each of the inner channels and blue-labelled GG in one of the outer channels, while a second outer channel is blocked (FIG. 1D). The resulting fibres, depicted in FIG. 8D, are composed by three individual compartments, where the less viscous hydrogel precursor forms a continuous shell. This shell surrounds the hydrogel compartments obtained from the hydrogel precursors with less viscosity. The core is made of GG, the precursor with intermediate viscosity, while the in-between compartment is made of red-labelled GG.

By alternating the materials of the previous embodiment of ribbon configuration (FIG. 1E), it is possible to fabricate fibres with four distinct compartments (red-blue-red-blue or blue-red-blue-red), named double-Janus (FIG. 8E), inspired by the typical two-compartment Janus fibres described elsewhere.

A double core-shell shape is obtained with a slightly distinct chip (Flow Focusing, Dolomite), that does not have the 3D geometry and only has three microfluidic channels. A double core-shell fibre is obtained when using a flow condition which allows the inner material to go around and surround the outer one. To that end, the outer channels are filled with blue-labelled GG, while red-labelled GG flows in the inner channel. The resulting fibre has two compartments of blue GG, which are separated and covered by a third one made of red-labelled GG, as depicted in FIG. 8F.

### EXAMPLE 2

### Alginate-Based Fibres

In another embodiment, fibres can be fabricated with other materials that crosslink ionically (similarly to GG), as the widely used alginate (FIG. 10). For that, sodium alginate (Sigma) was dissolved overnight by stirring in water to a final concentration of 2 wt %. For contrasting and colouring, alginate was blended together with red or blue magnetic microparticles (screenMag, Chemicell), 1:10 particle dilution. From here, all steps taken were the same as those of the previous embodiment, for the preparation of GG fibres.

### EXAMPLE 3

### Fibres Prepared with Non-Ionic Materials

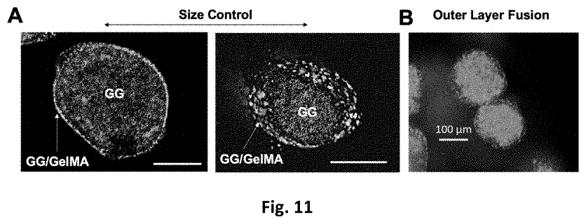

In an embodiment, non-ionic materials can be blended with ionic materials to be part of fibres' outer compartments. The outer compartments of the fibre must have an ionic crosslinking material as this will be responsible for giving immediate stability to the structure upon exiting the chip into the CaCl₂ bath. However, it is possible to blend other materials with the ionic crosslinking component in order to change the composition of the outer structure, e.g. the shell. A material such as Gelatin Methacryloyl (GelMA) can be blended with GG, allowing the fibre shape to be assured by the ionic component (FIG. 11A), but then also using a second crosslinking mechanism (UV) which crosslinks the GelMA also allowing the fusion of 2 fibres by crosslinking their shells (FIG. 11B). The Core-Shell shape is still possible since the GG/GelMA blend is less viscous than the inner GG.

In a form of the previous embodiment, gelatin methacryloyl Bloom 300 (Sigma) is dissolved at 5% in weight in water containing 0.25M Sucrose and 0.3 wt % Irgacure (Sigma). This was blended with a 0.5% GG solution at a 1:1 ratio and used as shell material. Upon forming the fibres through the ionic crosslinking by CaCl₂ 0.1M, the GelMA component was crosslinked using UV light ((320-500 nm) (Omnicure series 2000) for 50 seconds at 0.6 mW·cm⁻².

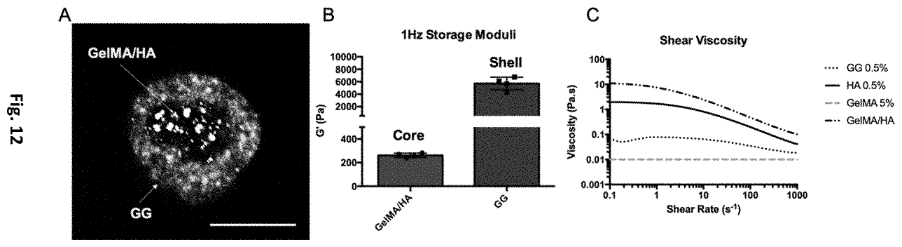

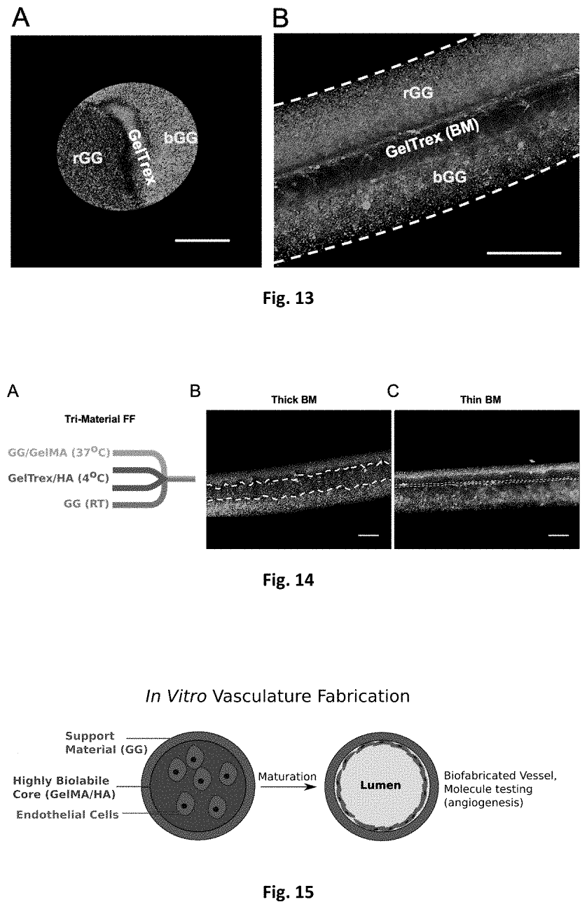

Non-ionic materials can also be included in the core surrounded by a GG shell, as showed in FIG. 12A. For that, it is necessary to manipulate the viscosity of the inner material to be at least 2-times higher than that of GG hydrogel precursor, used to form the shell. In that case, the required differences in viscosity are maintained and as such the core-shell structure can be formed. As an example, the UV crosslinkable GelMA, which would be too liquid and therefore impossible to place inside the core, can be dissolved in a high molecular weight hyaluronic acid solution (HA). For that, GelMA was dissolved to 5 wt. % in a solution of Hyaluronic Acid 0.5 wt % (Sodium Hyaluronate 1.5MDa, Lifecore) containing 0.25M sucrose and a3% Irgacure (Sigma). Differences in viscosity were studied using a Malvern Kinexus Pro+ Rheometer and a conical geometry, where the shear viscosity was recorded along a range of 0.1-1000 Hz (s⁻¹) of shear rate. Oscillation tests were also performed to characterize the mechanical properties (shear modulus) of GG (shell) and GelMA/HA (core) hydrogels. Briefly, an amplitude sweep was performed to derive the linear viscoelastic region, within which a frequency sweep was then performed to derive the storage (G′) and loss (G″) shear moduli of the hydrogels (FIG. 12B-C).

Since the hyaluronic solution is of high viscosity, the GelMA/HA blend overcomes the viscosity of GG and as such can flow as core surrounded by GG as shell (FIG. 12A). This allows to obtain a core which is fully independent from ionic crosslinking, being supported by the outer ionic-crosslinked shell. Fibres were spun into the CaCl₂ solution and afterwards exposed to UV light in order to crosslink the GelMA/HA core. To obtain fibres with a liquified core, the UV crosslinking step can be skipped.

In another embodiment, it is possible to introduce a thermal-crosslinking material within the ribbon-shape, i.e., surrounded by outer compartments of ionic crosslinking material, which will be separated by a ribbon of a third material. To approach the nature of tumour models, it is possible to include GelTrex (a Basement Membrane (BM) Derivative) gel separating two GG compartments (FIG. 9) by blending it with HA (increased viscosity). The GelTrex is stabilized within the fibre by the immediate GG crosslinking and some GelTrex/GG interaction but can then be further crosslinked by incubation at 37° C. (temperature).

In yet another embodiment, it is possible to combine the GelTrex ribbon with distinct materials flanking it. Therefore, one of the external compartments can be composed by a 1:1 GG/GelMa blend, instead of only GG, adding a third material to the structure, rendering tri-material ribbons (FIG. 14). UV crosslinking can be further used, as described in previous embodiments.

To obtain the BM structure within the 2 compartments, an overnight-thawed GelTrex Solution (Gibco) was mixed with the Hyaluronic Acid 0.5% solution in order to yield a final GelTrex concentration of at least 10 mg/mL according to the manufacturer's requirements for the formation of a gel by the GelTrex. To observe GelTrex within the outer Gellan Gum compartments, the Blue and Red Mags in the GG were uses and GelTrex was mixed with a 1:100 dilution of a 1% GG-FITC solution. For obtaining the tri-material structure, a similar approach was followed but instead of having GG 0.5% in both sides, one was replaced by a softer and more prone to remodelling GG/GelMA 1:1 blend. The distinct materials were kept at different temperatures during fabrication by maintaining the respective Eppendorf's at room temperature (GG), 37° C. (GelMA), and 4° C. (GelTrex/HA) to ensure fluidity within the chip. Fibres were collected in the CaCl₂ bath, then the GelMA component was crosslinked by quick UV exposure (previously described) and the GelTrex/HA was crosslinked by placing fibres in incubation at 37° C.

### EXAMPLE 4

### In Vitro Vasculature Fabrication

The core-shell flow focusing fibres can be used to include a soft, degradable material within a structurally stranger shell. Including endothelial cells in the core material allows for in vitro maturation and gradual organization in tubular-like structures (FIG. 15).

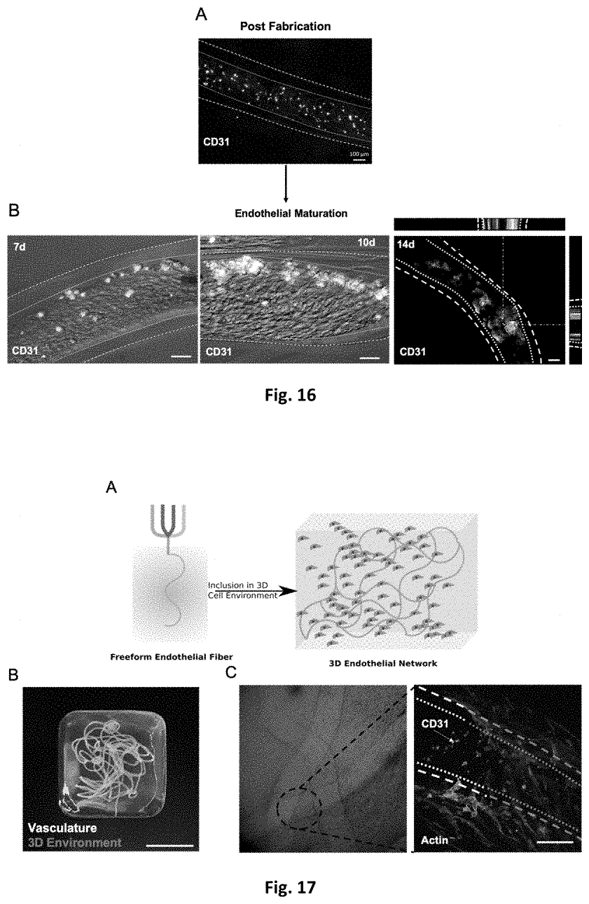

In an embodiment, human cermal microvascular endothelial cells were encapsulated inside a fibre with a GelMA/HA core (FIG. 16A), within which these were kept in culture, gradually arranging themselves towards lumen-like architectures (FIG. 16B). This approach therefore allows the recapitulation of vasculogenesis in vitro and can be used to model angiogenic responses.

To produce vascular structures, human dermal microvascular endothelial cells (hDMECs) were suspended in the core solution of GelMA/HA prior to the fibre fabrication at a density of 3×10⁶ cells·mL⁻¹. Fibres were spun into the CaCl₂ solution and afterwards exposed to UV light in order to crosslink the GelMA/HA core. To obtain fibres with a liquified core, the UV crosslinking step was skipped. Post-fabrication endothelial cell viability was assessed by incubation with medium containing 1:1000 dilution of Calcein AM (Thermofisher) and 1 μg/mL Propidium Iodide (Molecular Probes) for 30 mins at 37° C., 5% CO₂ and were then imaged under a fluorescent Axio Observer Inverted Microscope (Zeiss). For CD31 immunocytochemistry, fibres were fixed in formalin 10% for 10 mins at room temperature (RT), washed with PBS and incubated with 0.2% Triton X-100 (Thermo Fisher) in PBS for 12 minutes to enhance cell membrane permeability. After washing, non-specific interactions were blocked by incubating with 3% Bovine Serum Albumin (BSA) (Sigma) in PBS for 30 minutes. Afterwards, samples were incubated with the primary mouse CD31 antibody (DAKO, 1:50 dilution) in 1% BSA overnight at 4° C. These were then incubated with the secondary anti-mouse antibody (Alexa 488 donkey anti-mouse, Invitrogen, 1:500 dilution) for 1 hour at RT. At this stage, cell cytoskeleton and nuclei were also stained by adding phalloidin-TRITC (phalloidin—tetramethylrhodamine B isothiocyanate, Sigma) at 2 ug·mL⁻¹ and DAPI (4′,6-diamidino-2-phenylindole, Biotium) at 4 ug·mL⁻¹. Afterwards, samples were taken to image in the fluorescent microscope.

The obtained fibres can be also used to create free vascular structures that can be combined with distinct materials and cells, in order to approach more complex tissue engineering models (FIG. 17). These models can potentially inform about the interaction between different cells and the vascular structures existing in vivo, such as stroma/vasculature (fibroblasts/endothelial cells) or even tumour/vasculature (cancer cells/endothelial cells).

In order to integrate vascular core-shell fibres in the 3D environments, human dermal fibroblasts (hDFs) were suspended at a density of 3×10⁶ cells·mL⁻¹ in a 2 mg·mL⁻¹ solution of neutralized rat tail collagen type I (Gibco) (FIG. 17A). Core-shell endothelial fibres were randomly placed within well plates and the collagen-fibroblast solution was added to these and crosslinked by incubating at 37° C., resulting in the structure represented in FIG. 17B. After 3 days of culture, samples were fixed and stained with CD31, phalloidin-TRITC and DAPI, as previously described. To obtain 3D rendition of the environments, samples were imaged with a Z1 Light Sheet Microscope (Zeiss). Throughout this experiment, fibroblasts were cultured in complete alpha-MEM medium (Gibco), hDMECs were cultured in EGM-2 Bullet medium (Lonza) and co-cultures were kept with 1:1 mix of both media.

After just 3 days of culture, a complex collagen-fibroblast network fully surrounds the core-shell fibres with endothelial cells, thus representing an in vitro model of a vascularized tissue (FIG. 17C).

### EXAMPLE 5

### Complex 3D Cancer Models and Drug Testing

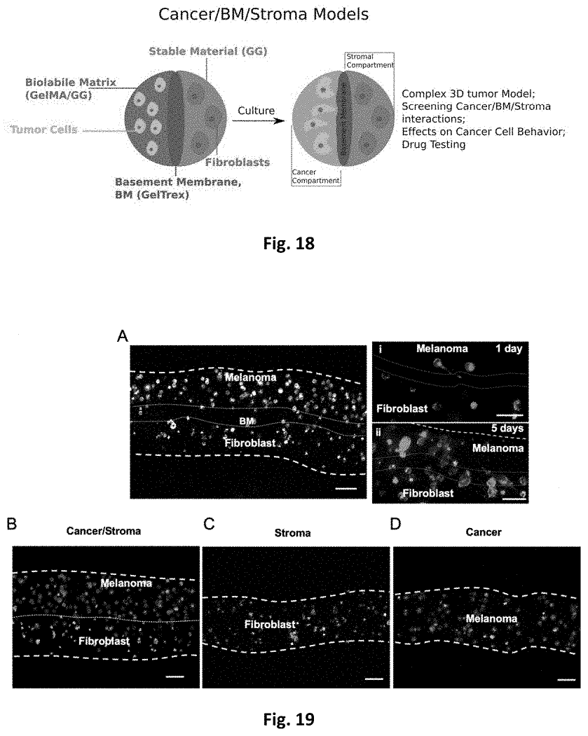

The ribbon shape (tri-material ribbon flow focusing) represents a unique platform to combine two different compartments with distinct environments and a third separating material within a same structure. This was used to fabricate complex 3D cancer models with one cancer compartment and one stromal compartment, separated by a Basement-membrane-(BM)-like ribbon, mimicking the first barrier cancer cells must overcome to metastasize, as schematically represented in FIG. 18.

Melanoma cells of the Sk-MEL-28 (ATCC) cell line were encapsulated in the GG/GelMA compartment and human dermal fibroblasts (hDFs) were encapsulated in the GG compartment, both representing the cancer and stroma, respectively, separated by the basement-membrane-mimicking GelTrex/HA. The degradable and adhesive GG/GelMA is used for the cancer compartment to allow cancer cells to move, while a more stable (GG) material is used for the stromal compartments, to keep fibroblasts in place and study mostly the cancer cell responses. In order to track the different cell types, these were stained with CellBrite Green and Orange (Biotium) according to the manufacturer's instructions. To fabricate the different modular fibres, showed in FIG. 19, it was only necessary to flow or stop flowing certain channels' materials, from all (Cancer/BM/Stroma) to only the side channels (Cancer/Stroma, no BM) or individual ones (Stroma- and Cancer-alone). With this model, melanoma cells were included in the cancer compartment, and dermal fibroblasts within the stromal compartment, both separated by a GelTrex BM in a unique melanoma-on-a-fibre structure. Fibres with tracked cells were observed under an Inverted Confocal Laser Scanning Microscope (Leica), throughout one week of culture.

Thus, it is possible to fabricate a ribbon structure with the inclusion of relevant cells such as melanoma (cancer) and fibroblasts (stroma) separated by a BM structure (GelTrex). As depicted in FIG. 19A, after 1 day of culture it is possible to observe melanoma cells protruding into the BM and clearly invading through it upon 5 days of culture (FIGS. 19Ai and Aii), which shows that the BM-invasive cancer responses can be recapitulated in this model. Moreover, the above discussed modular flow focusing configuration allows to obtain a cancer/stroma structure (FIG. 19B, no BM), single-stroma and single-cancer fibres, showed in FIGS. 19C and D, respectively. This allows not only screening responses on the complex model but to also understand the consequence of each distinct entity in the final outcome of cancer cell behaviour.

As a further embodiment, the cancer/BM/stroma modular platform can be used to test how the distinct compartments, and presence of the distinct entities, could impact the response of cancer cells to an anti-cancer drug (Doxorubicin).

For the drug tests, fibroblasts were stained blue prior to encapsulation using CellTracker blue CMAC Dye (7-amino 4-chloromethylcoumarin, Molecular Probes) according to manufacturer's instructions. These were then integrated in the fibres together with the cancer cells, and all modular fibres were produced. 24 h after fabrication, fibres were incubated with either culture medium (no treatment) or culture medium containing 1 μM of Doxorubicin (Carbosynth). One day after treatment, samples were incubated with medium containing 1:1000 dilution of Calcein AM (Thermofisher) and 1 μg/mL Propidium Iodide (Molecular Probes) for 30 mins at 37° C., 5% CO₂, for viability assessment. Images of the fibres were acquired in the Axio Observer inverted Microscope (Zeiss), and a cell profiler pipeline was used to derive the numbers of live and dead cells using the blue staining to distinguish fibroblasts from melanoma cells. Similarly, 2D controls were obtained by simply seeding the cells individually or in co-culture in 24 well-plates at a density of 20×10³ cells per well (10×10³ cells of each type in the case of co-cultures). 2D controls were then processed in the same way as the fibres. Throughout the experiments, fibroblasts were cultured in complete Minimum Essential Medium Eagle—alpha modification (α-MEM), melanoma cells in Eagle's minimum essential medium (EMEM, ATCC) and co-cultures with a 1:1 mix of both media.

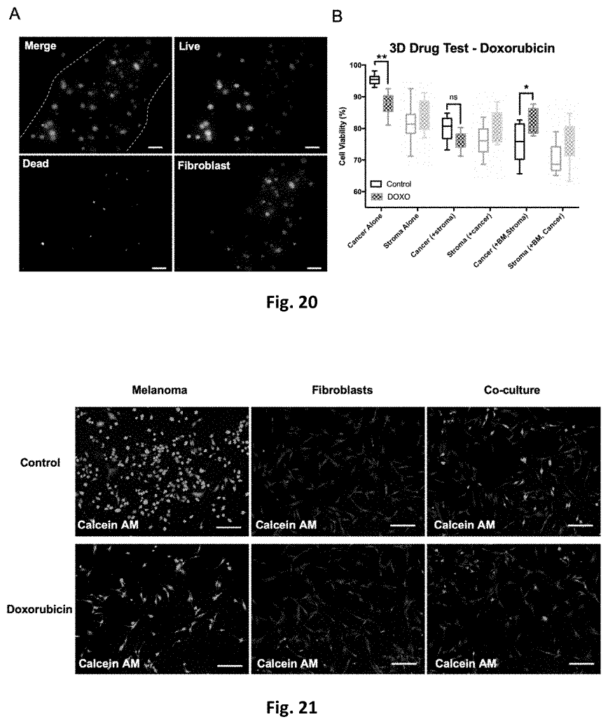

Using this configuration, it was possible to observe that the complexity of the model could lead to very different outcomes. Using markers for specific cell responses such as viability, together with cell trackers, it is possible to visualize the presence of death and live cells of each type. As showed in FIG. 20A, the cell number can be quantified in order to understand how drugs affect the viability of cancer cells in 3D environments of distinct complexity. The results plotted in FIG. 20B show that while cancer cells alone suffer a significant drop in viability after 24 h of doxorubicin treatment vs. the control, this is no longer significant in cancer/stroma combinations and, oppositely, when the full cancer/BM/stroma model is employed it is possible to see that the numbers of live cancer cells can improve upon treatment with Doxorubicin. This is evidence of how important it is to test the responses of cancer cells to drugs using models that can approach the live tissue structure and complexity, such as the one herein claimed. In fact, such type of screening is only possible with complex models, as the comparative data from 2D standards fail to provide similar information due to lack of tissue-like architectures (FIG. 21). Even if viable cancer and stromal cells can still be observed and followed in a 2D experiment, it is clear that the information on viability is lost as death cells detach and can no longer be considered. Also, due to the lack of any 3D architecture, the single cultures as well as the co-culture do not inform on any of the intricate interactions of cancer cells with 3D structures such as the basement membrane or with cells such as fibroblasts. The lack of shape and orientation fails to inform on anything other than eventual live cell numbers upon drug treatment.

### EXAMPLE 6

### All-in-One Tissue Engineering

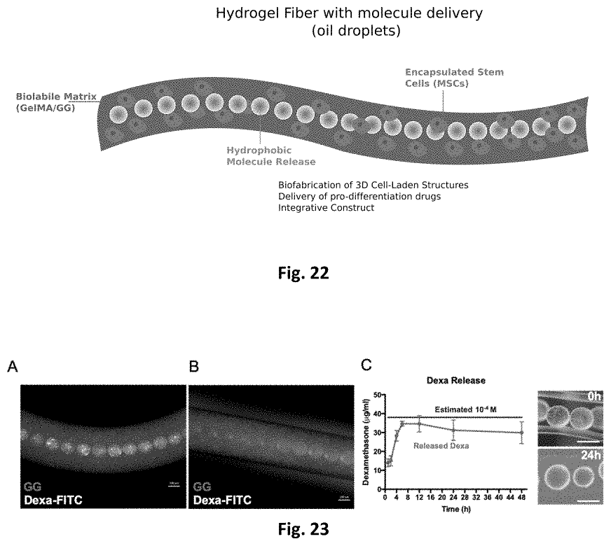

The oil-core hydrogel-shell structure was employed to fabricate an inclusive TE construct where cells/biomaterials can be combined with hydrophobic solutions containing pro-differentiation molecules (FIG. 22). As represented, the fabrication of such a structure can simultaneously transport the encapsulated stem cells with hydrophobic molecules that can be used to direct their differentiation.

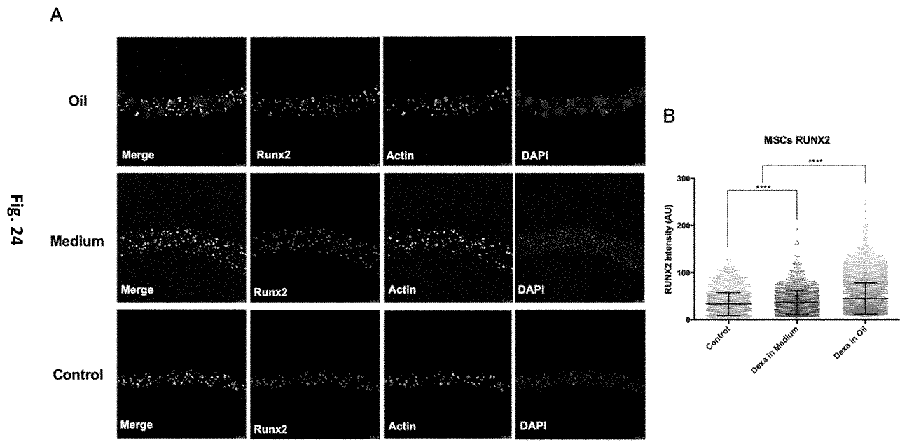

In an embodiment, it is possible to disperse dexamethasone, a hydrophobic molecule widely used in the differentiation protocols of stem cells. Frequently, dexamethasone has to be modified to be water-soluble and dissolved in medium. The use of oil-core hydrogel-shell fibres allows dexamethasone transportation within the oil in its pure form. The drug can then be released from the oil compartment to the surrounding environment, as showed in FIGS. 23A and B.

To visualize fibres with oil droplets containing dexamethasone (Dexamethasone), its fluorescent version (Dexamethasone-FITC, Molecular Probes) was dispersed in sesame oil by stirring, at a concentration of 0.25 mg·mL⁻¹. When a uniform dispersion was obtained, fibres were fabricated and imaged under the fluorescent Axio Observer Inverted Microscope (Zeiss).

To quantify the release of dexamethasone from the oil droplets, a dexamethasone standard curve was obtained using solutions of pure dexamethasone (Sigma) and its characteristic absorbance at 241 nm. Using this information, dexamethasone was dispersed in mineral oil at a concentration of 20 mg·mL⁻¹, estimated to yield a final concentration of 10⁻⁴M in 1 mL of phosphate buffered saline (estimated, upon total release from the oil), high enough for the instrument to be able to detect its gradual increase in concentration, measured through the 241 nm absorbance on a microplate reader (SYNERGY, Bio-tek instruments). The release was measured by keeping fibres in 6-well plates with 1 mL of PBS, and removing 100 μL of the well solution for measuring, replacing it with 100 μL of fresh PBS for up to 48 h. Brightfield images of the oil droplets were also acquired to observe the presence/absence of dispersed dexamethasone.

As plotted in FIG. 23C, the release of pure dexamethasone occurs for a period of around 12 h, gradually reaching the estimated values, considering oil droplet size, distribution and fibre length. It is possible to see that the dispersed dexamethasone present in the oil immediately post fabrication (0 h) is no longer visible after 1 day of incubation (24 h).

To evaluate the effect of dexamethasone in oil within the 3D fibres, bone marrow mesenchymal stem cells (MSCs) were encapsulated in 1:1 GG:GelMA hydrogel fibres containing oil droplets with pure dexamethasone and compared its effect to that of soluble dexamethasone in the medium or total absence of dexamethasone. For that, the cell-laden GG/GelMA fibres were cultured in normal medium, cultured in medium with 10⁻⁶M of water-soluble dexamethasone (dexamethasone in medium) or combined with oil droplets (oil) containing a dispersion of 0.5 mg·mL⁻¹ dexamethasone (estimated to release up to the same 10⁻⁶M). After 72 h in culture, cells were fixed and immunostained against Runx2 (Mouse Anti-Runx2 (Milipore), 1:300 dilution) and later incubated with Alexa 488 donkey-anti-mouse secondary antibody (1:500 dilution, Invitrogen) as well as with Phalloidin-TRITC (Sigma) 2 μg˜mL⁻³ and DAPI (Biotium) 4 μg·mL⁻¹. Cells were imaged and the Runx2 intensities quantified. Thousands of single-cell events among replicates were recorded.

After 3 days of culture, MSCs alone in normal medium (control), medium with soluble dexamethasone (medium) or together with oil droplets containing dexamethasone dispersion (oil) were stained against Runx2 together with actin and nuclei (DAPI) (FIG. 24A). Image quantification of thousands of single-cell Runx2 positive events show that there is a significant increase in the intensity of Runx2 signals by MSCs cultured with oil droplets releasing dexamethasone when compared to the presence of soluble dexamethasone in the medium, as plotted in FIG. 24B.

This test confirmed that this embodiment can not only replace the use of soluble dexamethasone in the medium but actually have a more powerful effect on the expression of the osteogenic differentiation marker Runx2, therefore validating the rationale behind oil-core hydrogel-shell fibres as all-in-one tissue engineering approaches.

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof. The above described embodiments are combinable.

The following claims further set out particular embodiments of the disclosure.

1. A multi-compartment hydrogel fibre wherein the fibre has an outer and an inner layer, the fibre comprising:
a first ionic hydrogel and a second component in a plurality of compartments,
wherein the second component is selected from the group consisting of: a second hydrogel, a hydrophilic solution, and a mixture thereof;
wherein the outer layer of the fibre comprises the first ionic hydrogel; and
the first ionic hydrogel and the second hydrogel have different compositions,
further wherein:
the plurality of compartments comprises two or more compartments that are axially nonconcentric/off-centred, or
the cross-section of one compartment of the plurality of the compartments is not circular, or
the fibre comprises an equivalent diameter less than 200 μm.

2. The hydrogel fibre according to claim 1, wherein the second component is the second hydrogel and wherein the compartments are axially nonconcentric/off-centred.

3. The hydrogel fibre of claim 1, wherein the fibre has an equivalent diameter less than 200 μm.

4. (canceled)

5. The hydrogel fibre of claim 1, wherein the first ionic hydrogel is selected from the group consisting of gellan gum, alginate, chitosan and mixtures thereof.

6. (canceled)

7. The hydrogel fibre of claim 1, wherein the second component is the second hydrogel and wherein the second hydrogel is selected from the group consisting of gellan gum, alginate, acid hyaluronic, gelatin, basement membrane extract, collagen, fibrin, biological lysates, silk solutions, dextran solutions, polyethylene glycol, chitosan, heparin, acrylamide, starch, cellulose, guar gum, xanthan gum and mixtures thereof.

8. The hydrogel fibre of claim 1, wherein the fibre is prepared by 3D flow-focusing.

9. The hydrogel fibre of claim 1, wherein the second component is the second hydrogel and wherein the second hydrogel is a photo-crosslinkable hydrogel.

10. The hydrogel fibre of claim 1 wherein the fibre comprises one or more additional ionic hydrogels, in one or more compartments of the plurality of compartments.

11. The hydrogel fibre of claim 10 wherein the one or more additional ionic hydrogels is a gellan gum hydrogel.

12. The hydrogel fibre of claim 11, wherein the fibre has a cross-sectional area that is in a shape of a core-shell, a ribbon, or a tricoaxial, or a double-Janus; or a double core-shell.

13. The hydrogel fibre of claim 12, wherein the plurality of compartments is axially concentric and the equivalent diameter is less than 200 μm.

14. The hydrogel fibre of claim 1 further comprising an anti-inflammatory agent, an antiseptic agent, an antipyretic agent, an anaesthetic agent, a therapeutic agent, a cell, or combinations thereof.

15. The hydrogel fibre of claim 14, wherein the cell is a non-human animal cell, or human cell, or stem cell, or combinations thereof.

16. A composition comprising the hydrogel fibre of claim 1 and a suitable carrier.

17. (canceled)

18. A kit comprising the hydrogel fibre of claim 1, wherein the kit is a multi-compartment medical-device selected from a cell carrier, therapeutic hydrogel, drug delivery depot, or combinations thereof.

19-21. (canceled)

22. A method to prepare the hydrogel fibre of claim 1 comprising:
injecting a first ionic hydrogel precursor and a second component solution into one or more channels of a flow focusing microfluidic chip, wherein the second component solution and the ionic hydrogel precursor have a distinct viscosity at 25° C.,
wherein the second component solution is selected from the group consisting of: a second hydrogel precursor, a hydrophilic solution, and a mixture thereof;
applying variable pressure to the one or more channels of the microfluidic chip using a pressure regulator to produce a hydrogel fibre precursor; and
extruding the hydrogel fibre precursor into an ionic cross-linking bath/solution to produce the hydrogel fibre.

23. The method of claim 22 wherein the ionic hydrogel precursor has a shear viscosity at 25° C. between 0.01 to 100 Pa·s.

24. The method of claim 23, wherein the second component solution is the second hydrogel precursor and the shear viscosity of the second hydrogel precursor is 2-1000 times higher than the shear viscosity of the first ionic hydrogel precursor.

25. The method of claim 22, wherein the flow focusing microfluidic chip comprises a plurality of channels.

26. The method of claim 22, wherein the pressure applied in one channel of the one or more channels is independent to the pressure applied in another channel of the one or more channels.

27. The method of claim 22, wherein the pressure to the one or more channels is in the range of from 10 to 800 kPa.

28-33. (canceled)
